(19) United States
(12) Patent Application Publication
(10) Pub. No.: US 2017/0224853 A1
(43) Pub. Date: Aug. 10, 2017

(54) GERMICIDAL IRRADIATION PUSH-BUTTON SYSTEMS AND METHODS

(71) Applicant: Matthew Jay, Basalt, CO (US)

(72) Inventor: Matthew Jay, Basalt, CO (US)

(21) Appl. No.: 15/428,820

(22) Filed: Feb. 9, 2017

Related U.S. Application Data
(60) Provisional application No. 62/293,140, filed on Feb. 9, 2016.

(51) Int. Cl. A61L 2/10; A61L 2/26

(52) U.S. Cl. CPC A61L 2/10; A61L 2/26

(57) ABSTRACT

Germicidal irradiation push-button systems for disinfecting surface(s) of push-button(s). In an aspect, a germicidal irradiation push-button system comprises a push-button having a first surface recessed behind a panel, wherein the panel has an opening for accessing the first surface; an ultraviolet (UV) light source located behind the panel for irradiating the first surface; and a UV-light control module for controlling the UV-light source.

### RELATED APPLICATIONS

This application claims priority to U.S. patent application Ser. No. 62/293,140, titled “Germicidal Irradiation Push Button” and filed Feb. 9, 2016, which is hereby incorporated by reference in its entirety.

### BACKGROUND

Surfaces designed for frequent touching in public provide potential germ transmission between persons, which may spread disease. Irradiation with ultraviolet light is one method of disinfecting surfaces to reduce germ transmission. However, ultraviolet light exposure is harmful to human skin and eyes and is therefore often not appropriate for use in public settings.

### BRIEF DESCRIPTION OF THE FIGURES

FIG. 1 schematically illustrates one system for a germicidal irradiation push-button, in an embodiment.

FIG. 2 schematically illustrates a germicidal irradiation push-button, in an embodiment.

FIG. 3 is a block diagram of a germicidal irradiation push-button system, in an embodiment.

FIG. 4 is a flow diagram of one method for germicidal irradiation of the push-button of FIG. 3, in an embodiment.

### DETAILED DESCRIPTION OF THE EMBODIMENTS

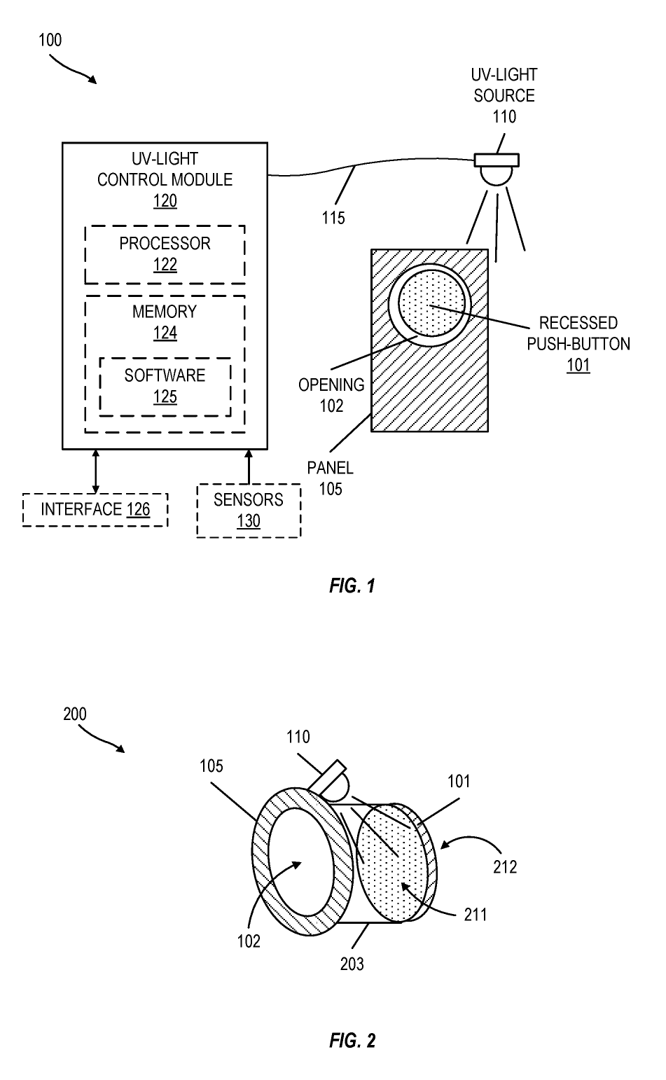

FIG. 1 schematically illustrates an exemplary system 100 for a germicidal irradiation push-button. System 100 provides an ultraviolet (UV) light source 110 that irradiates germs on a push-button 101 recessed behind panel 105. The recessed push-button 101 is accessed for example by a user's finger via an opening 102 through panel 105. Although FIG. 1 shows one recessed push-button 101, system 100 may have more than one recessed push-button 101, each with an opening 102 or multiple push-buttons 101 within a given opening 102, without departing from the scope hereof. In an embodiment, panel 105 is located within an enclosed space, such as an elevator, and recessed push-button 101 enables selection of a destination floor.

UV-light source 110 emits UV-C type electromagnetic radiation at or near a wavelength of 260 nm, in certain embodiments. Examples of UV-light source 110 include but are not limited to a mercury-vapor lamp or a UV-light-emitting diode (LED). By recessing push-button 101 behind panel 105, UV-light exposure is reduced outside of panel 105.

Control of UV-light source 110 is provided by UV-light control module 120. In an embodiment, UV-light control module 120 is operable in response to a signal from a simple switch that allows UV-light source 110 to be manually turned on or off by a user. Examples of a switch include a toggle switch, a pushbutton switch, a selector switch, a pressure switch, inductive switch, foot switch, pull switch, and dimmer switch. In another embodiment, UV-light control module 120 includes a processor 122, a memory 124, a software 125, and a local or remote interface 126 coupled to UV-light source 110 via communication path 115, which may include one or both of a wired and/or a wireless communication media. Examples of a local or remote interface 126 include buttons or touch screen as part of a wireless device such as a mobile phone or as part of a device connected by wire to UV-light control module 120. Examples of wired communication media include copper or aluminum wiring, shielded or unshielded wiring, cable, such as telephone, Ethernet, coaxial, or triaxial, and fiber-optic cable, or any other known wired communication protocols. Examples of wireless communication media include radio or any radio-frequency communication protocols, cellular, Bluetooth, any microwave-frequency communication protocols, or any other known wireless communication protocols. Memory 124, in some embodiments, is a memory system that includes both transitory memory such as RAM and non-transitory memory such as, ROM, EEPROM, Flash-EEPROM, magnetic media including disk drives, optical media. Memory 124 stores software 125 as machine readable instructions executable by processor 122 to process data from sensors 130. UV-light control module 120 controls UV-light source 110 as illustrated in FIG. 4 and described below. For example, UV-light control module 120 may determine if one or more persons are within a space, such as an elevator. UV-light source 110 may be safely illuminated to provide germicidal irradiation to recessed push-button 101 if no one is in the space, and optionally if a door to the space is closed, as described in FIG. 4 and below.

System 100 may further include one or more sensors 130 to determine for example if any person is nearby for safe operation of UV-light source 110. Thus, system 100 may be disposed in a hallway, for example, outside of an elevator, for example, for safe germicidal irradiation of an elevator call button, with UV-light control module 120 determining via sensors 130 that the hallway is unoccupied. Sensors 130 may include infra-red, near-infrared, optical image, acoustic-, or microwave-based motion detection, or any other known motion detection method, for identifying whether or not the hallway is unoccupied.

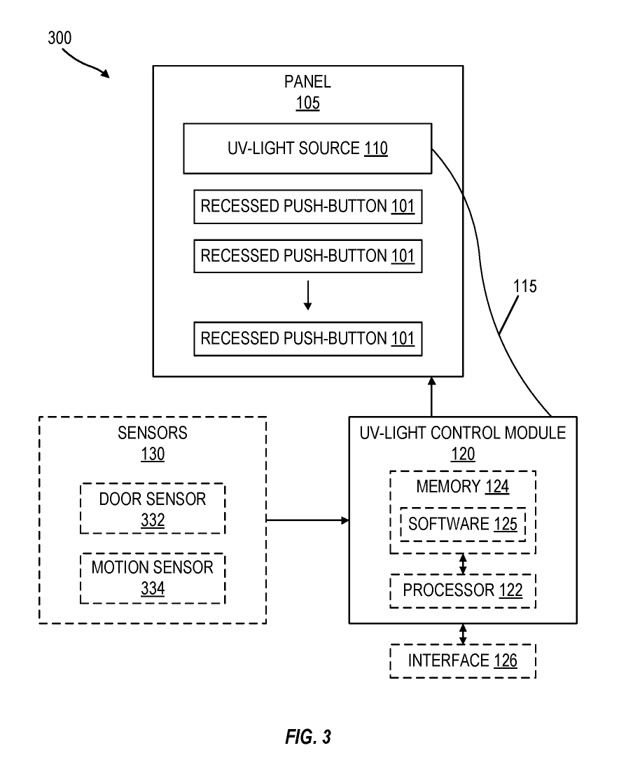

FIG. 2 schematically illustrates an exemplary germicidal irradiation push-button 200. Germicidal irradiation push-button 200 includes recessed push-button 101, FIG. 1, which is recessed behind panel 105 via a recess distance 203. Recess distance 203 may include a window for transmitting UV-light, such as an air gap or a UV-transparent material such as quartz or other natural or synthetic material. Only a portion of panel 105 is shown to allow viewing of UV-light source 110 and recessed push-button 101. Note that FIG. 2 is not drawn to scale. In particular, recess distance 203, which is exaggerated in FIG. 2 for clarity of illustration, is sized to allow a user to easily touch a first surface 211 of push-button 101 with, for example, a finger.

UV-light source 110 may be angled towards first surface 211, as shown in FIG. 2, to increase an incidence of UV-light reaching first surface 211. Only one UV-light source 110 is depicted in FIG. 2 for clarity of illustration, but more than one UV-light source 110 may be used without departing from the scope hereof. In an alternative embodiment, UV-light source 110 is located behind a second surface 212 of recessed push-button 101, and recessed push-button 101 is made of a UV-transparent material, such as quartz or other natural or synthetic material, to allow transmission of UV-C light for irradiating germs on first surface 211. In another embodiment, one or more reflective surfaces such as mirrors are positioned behind panel 105 to angle UV-light towards first surface 211 for increasing the incidence of UV-light thereupon, either in combination with or independently of a UV-transparent push-button 101. Thus, various arrangements of UV-light source(s) with or without reflective surfaces are possible without departing from the scope hereof.

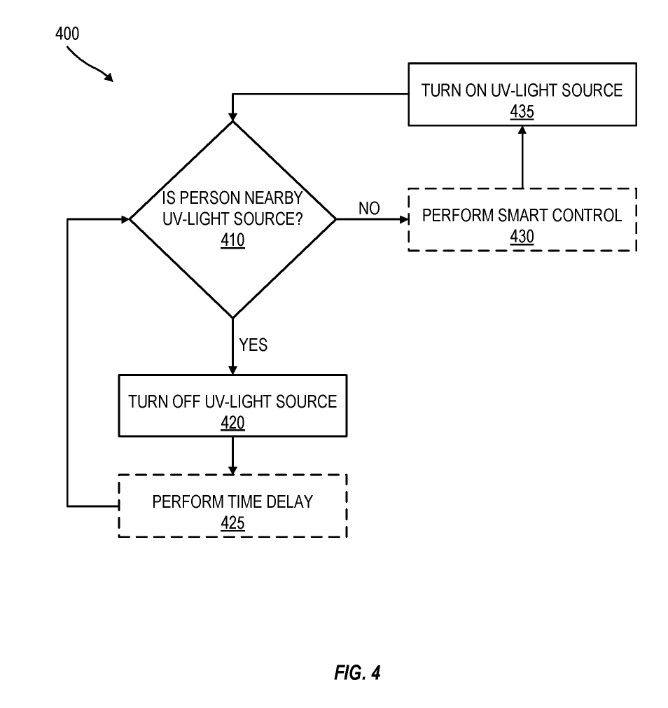

FIG. 3 is a block diagram of an exemplary germicidal irradiation push-button system 300, which is an example of system 100, FIG. 1. System 300 includes panel 105 having UV-light source 110 and a plurality of recessed push-buttons 101, as well as UV-light control module 120, FIG. 1. UV-light control module 120 is communicatively coupled to UV-light source 110 and the plurality of recessed push-buttons 101 within panel 105. System 300 optionally includes one or more sensors for sensing a presence of one or more persons nearby, such as for example a door sensor 332 and/or a motion sensor 334. Door sensor 332 is for example a magnetic switch or a reed switch mechanically coupled to a door and communicatively coupled to UV-light control module 120 via communication path 115, FIG. 1. Motion sensor 334 is for example an optical (e.g., infrared)-, acoustic- or microwave-based sensor that determines motion of an object or person nearby. In an embodiment, panel 105 is disposed within a space and UV-light control module 120 determines whether a door to the space is open via door sensor 332 and whether a person or object is moving or has recently moved inside the space via motion sensor 334. In an embodiment, the space is within an elevator.

FIG. 4 is a flow diagram of an exemplary method for germicidal irradiation of a push-button. Method 400 is used to safely irradiate germs on recessed push-button 101, FIGS. 1-3 for example, in the absence of any nearby persons.

In a step 410, a decision is made as to whether any person is nearby a UV-light source positioned to germicidally irradiate a recessed push-button. If in step 410, a person is determined to be nearby the UV-light source, method 400 proceeds to step 420 to turn off the UV-light source. Otherwise, method 400 proceeds to step 430. In an example of step 410, UV-light control module 120 determines whether any person is nearby UV-light source 110 by sensors 130, FIGS. 1, 3.

In step 420, the UV-light source is turned off after determining that a person may be nearby. In an example of step 420, UV-light source 110 is turned off after UV-light control module 120 determines via sensors 130 that a person is nearby. After step 420, method 400 proceeds to optional step 425.

In an optional step 425, a time delay occurs. In an example of optional step 425, UV-light control module 120 performs a time delay prior to returning to step 410 as a safety precaution to ensure that no person is, or has recently been, nearby UV-light source 110.

In an optional step 430, a smart control of UV-light source is performed prior to proceeding to step 435. In an example of optional step 430, UV-light control module 120 performs smart control of UV-light source 110 via commands of software 125 in memory 124, executed by processor 122. An example command is a time command that may be used to delay turning on UV-light source 110 until a predetermined time, such as overnight, to reduce potential exposure to users. Another example command is a frequency command, which might forego any delay and proceed immediately to step 435 during periods of more frequent use of recessed push-button 101, in order to reduce the spread of germs.

In step 435, the UV-light source is turned on after determining that no one is nearby. In an example of step 435, UV-light source 110 is turned on after UV-light control module 120 determines via sensors 130 an absence of any persons nearby.

Method 400 repeats continuously to safely provide germicidal irradiation of recessed push-button 101. In an embodiment, sensors 130 include door sensor 332 and motion sensor 334, FIG. 3 for determining whether a door to an enclosed space, such as an elevator door, is open and whether motion is detected within the enclosed space, such as an elevator, respectively. By repeatedly determining whether the elevator door is not open and/or motion is not detected, method 400 continuously monitors an absence of any persons nearby in order to safely irradiate germs using UV-light source 110.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which might be said to fall therebetween.

What is claimed is:

1. A germicidal irradiation push-button system, comprising:
a push-button having a first surface recessed behind a panel, wherein the panel has an opening for accessing the first surface;
an ultraviolet (UV) light source located behind the panel for irradiating the first surface; and
a UV-light control module for controlling the UV-light source.

2. The germicidal irradiation push-button system of claim 1, further comprising one or more sensors, the UV-light control module determining an absence or a presence of any persons nearby the push-button based on the one or more sensors, and controls the UV-light source in response to determination of the absence of any persons nearby the push-button to provide germicidal irradiation.

3. The germicidal irradiation push-button system of claim 1, further comprising one or more reflective surfaces for reflecting UV-light from the UV-light source to the first surface.

4. The germicidal irradiation push-button system of claim 1, the push-button further having a second surface, opposite the first surface, and the UV-light source is positioned behind the second surface.

5. The germicidal irradiation push-button system of claim 4, the push-button comprising a UV-transparent material, such that UV-light passes through the push button for irradiation of the first surface from the UV-light source located behind the second surface.

6. The germicidal irradiation push-button system of claim 1, the UV-light control module operable in response to a signal from a switch for local or remote operation of the UV-light source.

7. The germicidal irradiation push-button system of claim 1, the UV-light control module being connected to the UV-light source by a communication path.

8. The germicidal irradiation push-button system of claim 7, the communication path includes one or both of a wired and/or a wireless communication media.

9. The germicidal irradiation push-button system of claim 1, the UV-light control module includes an interface for local or remote operation of the UV-light source.

10. A method for germicidal irradiation of a push-button comprising:
determining whether any person is nearby an ultraviolet (UV) light source, wherein the UV-light source is positioned to germicidally irradiate a recessed surface of the push-button;
turning on the UV-light source after determining that no person is nearby; and
turning off the UV-light source after determining that a person is nearby.

11. The method of claim 10, further comprising determining whether any person is nearby the UV-light source by determining presence of motion with a motion sensor.

12. The method of claim 10, further comprising determining whether any person is within a space nearby the UV-light source by determining, with a door sensor, whether a door to the space is open or closed.

13. A germicidal irradiation push-button system comprising:
a push-button having a first surface recessed behind a panel, wherein the panel has an opening for accessing the first surface;
an ultraviolet (UV) light source located behind the panel for irradiating the first surface; and
a UV-light control module for controlling the UV-light source, the UV-light control module comprising:
a memory, a software stored in the memory, the software comprising machine readable instructions, and a processor for executing the software.

14. The germicidal irradiation push-button system of claim 13, further comprising one or more sensors, the UV-light control module determining an absence or a presence of any persons nearby the push-button based on the one or more sensors, for controlling the UV-light source to provide germicidal irradiation.

15. The germicidal irradiation push-button system of claim 13, the UV-light control module being connected to the UV-light source by a communication path.

16. The germicidal irradiation push-button system of claim 13, the UV-light control module including a switch for local or remote operation of the UV-light source.

17. The germicidal irradiation push-button system of claim 13, the UV-light control module including an interface for local or remote operation of the UV-light source.

18. The germicidal irradiation push-button system of claim 13, further comprising one or more reflective surfaces for reflecting UV-light from the UV-light source to the first surface of the push-button.

19. The germicidal irradiation push-button system of claim 13, the push-button having a second surface, opposite the first surface, and the UV-light source is positioned behind the second surface.

20. The germicidal irradiation push-button system of claim 13, the push-button comprising a UV-transparent material, such that UV-light passes through the push button for irradiation of the first surface from the UV-light source located behind the second surface.
